SEQUENCE LISTING (42 sequences)

SEQ ID NO: 1 - protein, 9 aa, Artificial Sequence
  Description of Artificial Sequence Synthetic peptide
CXGPXTWXC

SEQ ID NO: 2 - protein, 12 aa, Artificial Sequence
  Description of Artificial Sequence Synthetic peptide
YXCXXGPXTWXC

SEQ ID NO: 3 - protein, 16 aa, Artificial Sequence
  Description of Artificial Sequence Synthetic peptide
XYXCXXGPXTWXCXXX

SEQ ID NO: 4 - protein, 20 aa, Artificial Sequence
  Description of Artificial Sequence Synthetic peptide
GGLYLCRFGPVTWDCGYKGG

SEQ ID NO: 5 - protein, 20 aa, Artificial Sequence
  Description of Artificial Sequence Synthetic peptide
GGTYSCHFGPLTWVCKPQGG

SEQ ID NO: 6 - protein, 18 aa, Artificial Sequence
  Description of Artificial Sequence Synthetic peptide
GGTYSCHFGPLTWVCKPQ

SEQ ID NO: 7 - protein, 20 aa, Artificial Sequence
  Description of Artificial Sequence Synthetic peptide
GGDYHCRMGPLTWVCKPLGG

SEQ ID NO: 8 - protein, 20 aa, Artificial Sequence
  Description of Artificial Sequence Synthetic peptide
VGNYMCHFGPITWVCRPGGG

SEQ ID NO: 9 - protein, 20 aa, Artificial Sequence
  Description of Artificial Sequence Synthetic peptide
GGNYMCHFGPITWVCRPGGG

SEQ ID NO: 10 - protein, 20 aa, Artificial Sequence
  Description of Artificial Sequence Synthetic peptide
GGVYACRMGPITWVCSPLGG

SEQ ID NO: 11 - protein, 19 aa, Artificial Sequence
  Description of Artificial Sequence Synthetic peptide
VGNYMAHMGPITWVCRPGG

SEQ ID NO: 12 - protein, 18 aa, Artificial Sequence
  Description of Artificial Sequence Synthetic peptide
GGPHHVYACRMGPLTWIC

SEQ ID NO: 13 - protein, 18 aa, Artificial Sequence
  Description of Artificial Sequence Synthetic peptide
GGTYSCHFGPLTWVCKPQ

SEQ ID NO: 14 - protein, 20 aa, Artificial Sequence
  Description of Artificial Sequence Synthetic peptide
GGLYACHMGPMTWVCQPLRG

SEQ ID NO: 15 - protein, 22 aa, Artificial Sequence
  Description of Artificial Sequence Synthetic peptide
TIAQYICYMGPETWECRPSPKA

SEQ ID NO: 16 - protein, 13 aa, Artificial Sequence
  Description of Artificial Sequence Synthetic peptide
YSCHFGPLTWVCK

SEQ ID NO: 17 - protein, 11 aa, Artificial Sequence
  Description of Artificial Sequence Synthetic peptide
YCHFGPLTWVC

SEQ ID NO: 18 - protein, 20 aa, Artificial Sequence
  Description of Artificial Sequence Synthetic peptide
GGTYSCHFGPLTWVCKPQGG

SEQ ID NO: 19 - protein, 14 aa, Artificial Sequence
  Description of Artificial Sequence Synthetic peptide
GGCRIGPITWVCGG

SEQ ID NO: 20 - protein, 22 aa, Artificial Sequence
  Description of Artificial Sequence Synthetic peptide
LGRKYSCHFGPLTWVCQPAKKD

SEQ ID NO: 21 - protein, 20 aa, Artificial Sequence
  Description of Artificial Sequence Synthetic peptide
GGTASCHFGPLTWVCKPQGG

SEQ ID NO: 22 - protein, 20 aa, Artificial Sequence
  Description of Artificial Sequence Synthetic peptide
GGNYYCRFGPITFECHPTGG

SEQ ID NO: 23 - protein, 20 aa, Artificial Sequence
  Description of Artificial Sequence Synthetic peptide
GGEYLCRMGPMTWVCTPVGG

SEQ ID NO: 24 - protein, 20 aa, Artificial Sequence
  Description of Artificial Sequence Synthetic peptide
GGLYTCRMGPITWVCLPAGG

SEQ ID NO: 25 - protein, 20 aa, Artificial Sequence
  Description of Artificial Sequence Synthetic peptide
GGTTSCHFGPLTWVCKPQGG

SEQ ID NO: 26 - protein, 20 aa, Artificial Sequence
  Description of Artificial Sequence Synthetic peptide
GGTFSCHFGPLTWVCKPQGG

SEQ ID NO: 27 - protein, 20 aa, Artificial Sequence
  Description of Artificial Sequence Synthetic peptide
GGTYSCHFGALTWVCKPQGG

SEQ ID NO: 28 - protein, 20 aa, Artificial Sequence
  Description of Artificial Sequence Synthetic peptide
GGTYSCHFGPLAWVCKPQGG

SEQ ID NO: 29 - protein, 20 aa, Artificial Sequence
  Description of Artificial Sequence Synthetic peptide
GGTYSCHFAPLTWVCKPQGG

SEQ ID NO: 30 - protein, 20 aa, Artificial Sequence
  Description of Artificial Sequence Synthetic peptide
GGTYSCHFGPATWVCKPQGG

SEQ ID NO: 31 - protein, 20 aa, Artificial Sequence
  Description of Artificial Sequence Synthetic peptide
GGTYSCHFGPLTAVCKPQGG

SEQ ID NO: 32 - protein, 20 aa, Artificial Sequence
  Description of Artificial Sequence Synthetic peptide
GGTYSCHFGPLTFVCKPQGG

SEQ ID NO: 33 - protein, 16 aa, Artificial Sequence
  Description of Artificial Sequence Synthetic peptide
TYSCHFGPLTWVCKPQ

SEQ ID NO: 34 - protein, 14 aa, Artificial Sequence
  Description of Artificial Sequence Synthetic peptide
YSCHFGPLTWVCKP

SEQ ID NO: 35 - protein, 12 aa, Artificial Sequence
  Description of Artificial Sequence Synthetic peptide
SCHFGPLTWVCK

SEQ ID NO: 36 - protein, 19 aa, Artificial Sequence
  Description of Artificial Sequence Synthetic peptide
GGTYSCFGPLTWVCKPQGG

SEQ ID NO: 37 - protein, 18 aa, Artificial Sequence
  Description of Artificial Sequence Synthetic peptide
TYSCHFGPLTWVCKPQGG

SEQ ID NO: 38 - protein, 12 aa, Artificial Sequence
  Description of Artificial Sequence Synthetic peptide
YSCHFGPLTWVC

SEQ ID NO: 39 - protein, 8 aa, Artificial Sequence
  Description of Artificial Sequence Synthetic peptide
HFGPLTWV

SEQ ID NO: 40 - protein, 13 aa, Artificial Sequence
  Description of Artificial Sequence Synthetic peptide
YSCHFGALTWVCK

SEQ ID NO: 41 - protein, 20 aa, Artificial Sequence
  Description of Artificial Sequence Synthetic peptide
GGTYSEHFGPLTWVKKPQGG

SEQ ID NO: 42 - protein, 20 aa, Artificial Sequence
  Description of Artificial Sequence Synthetic peptide
GGTYSCHFGPLTWVCKPQGG

(19) United States
(12) Patent Application Publication
(10) Pub. No.: US 2008/0249001 A1
(43) Pub. Date: Oct. 9, 2008

(54) AGENTS THAT ALLEVIATE SIDE-EFFECTS CAUSED BY CHEMOTHERAPY AGENTS

(75) Inventors: Akiko Onuki, Kawasaki-shi (JP); Kazumi Tashiro, Kawasaki-shi (JP); Yuzuru Eto, Kawasaki-shi (JP); Tomoyuki Konda, Kawasaki-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: AJINOMOTO CO. INC., Tokyo (JP)

(21) Appl. No.: 11/924,080

(22) Filed: Oct. 25, 2007

Related U.S. Application Data
(60) Provisional application No. 60/912,522, filed on Apr. 18, 2007.
(60) Provisional application No. 60/854,103, filed on Oct. 25, 2006.

(30) Foreign Application Priority Data
Oct. 25, 2006 (JP) 2006-290162
Apr. 18, 2007 (JP) 2007-109220

(51) Int. Cl. A61K 38/14; C07C 53/00; A61K 31/195; A61P 35/00

(52) U.S. Cl. 514/8; 562/560; 514/561

(57) ABSTRACT

The present invention provides improving agents of side-effects of chemotherapy agents. Though the erythroid progenitor cells decrease due to the side-effect of chemotherapy agents, such side-effect can be improved by chemotherapy agents which comprise arginine as the active ingredient.

### TECHNICAL FIELD OF THE INVENTION

The present invention relates to improving agents of side-effects of chemotherapy agents, which comprise arginine.

### BACKGROUND OF THE INVENTION

Side-effects are problems in the cancer treatment with administering chemotherapy agents through or by intravenous drip. Though the development of cancer chemotherapy with chemotherapy agents has enhanced the therapeutic effect thereof, many of the chemotherapy agents also affect normal cells and a selective agent which affects cancer cells only has not yet been developed.

Patent Literature 1 discloses that a physiologically active substance, lisoserine, which is produced from one of the strains belonging to actinomycete, and the analogues obtained by chemically inducing them have the inhibiting action of transcribing human cyclin A gene and the cell-cycle arresting action of mammal's cells; and they also have the action of preventing or diminishing myelosuppression or acomia caused by etoposide that is an antitumor agent.

Patent Literature 2 discloses the method for increasing the dosage of a topoisomerase inhibitor that can be safely and effectively administered to patients; and said method which comprises the step of administering thalidomide in the amount which diminishes the side-effects related to the administration of the topoisomerase inhibitor to the patients who are necessary to increase the dosage.

Thus, various combination drugs have been examined in order to diminish the side-effects of chemotherapy agents. The most major side-effect that should be improved is myelopoietic suppression, and it is thought to occur because of the cell damage to hematopoietic cells that exist in the bone marrow. Further, toxicity to kidney is also serious, and the decrease of EPO production occurs as a result of the impaired renal function. From these causes, anemia becomes the serious side-effect that accompanies cancer chemotherapy. Since such side-effect deteriorates the QOL (quality of life) of patients, makes them deplete mentally and physically, and finally affects the cancer treatment, it is thought to be the important problem to improve anemia that accompanies the administration of chemotherapy agents. At present, it is desired to develop a therapeutic drug for directly and safely improving the side-effects of chemotherapy in various treatments, and more specifically myelosuppression, particularly toxicity to myelopoietic cells.

Generally, in the treatment of anemia, erythroid hematopoiesis is an essential process for homeostatic sustainment of the number of erythrocytes. The average human erythroid lifespan is about 120 days. Since senescent erythrocytes are continuously removed from the circulating system, about 100 billion erythrocytes are newly produced in the body of adults every day. The production of erythrocytes are thoroughly studied and described in numbers of descriptions. As a typical example, the following brief summary is excerpted from “Ketsuekigaku (Hematology), Chugai Igakusha”:

In the bone marrow, there are multipotent stem cells which can differentiate into various blood cell series, and a part of the multipotent stem cells differentiate into erythroid progenitor cells that are determined to differentiate into the erythroid series. The youngest cells that are identified as the erythroid progenitor cells are BFU-E (burst forming unit-erythroid) and more differentiative CFU-E (colony forming unit-erythroid). After CFU-E, the cells divide and differentiate into proerythroblasts, basophilic erythroblasts, polychromatic erythroblasts, and orthochromatic erythroblasts. Then, they are denucleated to mature from reticulocytes into erythrocytes. Reaching the phase of BFU-E and CFU-E, the differentiation into the erythroid series is definitely determined, and the cells do not differentiate into other blood cell series other than erythrocytes. Therefore, when the number of BFU-E and CFU-E increases, erythroid hematopoiesis is promoted.

The homeostasis of erythroid hematopoiesis is mainly sustained by erythropoietin (EPO) that is a hematopoietic factor. EPO is mainly produced in the kidney, circulates in the blood, acts on CFU-E in the bone marrow, and promotes the erythroid hematopoiesis by stimulating the proliferation and differentiation of CFU-E. When EPO is not produced to a normal level and becomes deficient, or when myelopoiesis is inhibited by the side-effects of chemotherapy agents, CFU-E decreases and erythroid hematopoiesis weakens to develop anemia. Anemia is a diseased state in which the requirement of the body for transporting oxygen cannot be met due to the deficient hemoglobin concentration. Since anemia has clinical symptoms such as the lowered motivation to work, fatigability, breathlessness, lightheadedness and palpitation, the improvement of such symptoms is desired.

Non-patent Literature 1 discloses that hemoglobin and the number of erythrocytes increased by repeatedly administering arginine to normal rats. However, the action mechanism thereof is unclear, that is, whether it is the effect of enhancing the erythroid neogenesis (hematopoiesis) or the effect of enhancing the erythroid lifespan. Further, it does not either disclose for what kind of anemia such administration of arginine is useful or whether arginine relates to the solution for diminishing the side-effects of chemotherapy agents.

Non-patent Literature 2 discloses that the anemic condition of patients with renal anemia was improved (the number of erythrocytes became close to the normal level) by administering arginine to them; and that such action comes from the action mechanism wherein arginine promotes renal EPO production. However, the Literature 2 as well as non-patent Literature 3 does not disclose the effect of diminishing myelosuppression caused by the administration of chemotherapy agents.

Though Non-patent Literature 3 discloses the existence of a transporter, the search results thereof are not the one wherein the myelopoietic action of arginine is expected.

In Non-patent Literature 4, the effect of arginine on cancer metastasis is examined, and the improving effect of the myelocytic toxicity caused by the side-effects of cancer chemotherapy cannot be predicted from the action mechanism reached from the thesis results.

Thus, the non-patent literatures disclose the EPO production promoting action of arginine, but any of them does not focus on the effect of arginine on diminishing the side-effects of chemotherapy agents and, particularly, they do not indicate that arginine diminishes the toxicity to erythroid progenitor cells.

### [Patent Literature 1] JP 2003-128581 A

### [Patent Literature 2] JP 2003-533484 A

[Non-patent Literature 1] Int. J. Toxicol., 2004, 23; 101-105

### [Non-patent Literature 2] IGAKU NO AYUMI (Journal of Clinical and Experimental Medicine) 2004, 211; 839-840

### [Non-patent Literature 3] Blood., 2006, 107; 1352-1356

[Non-patent Literature 4] Annals of nutrition & metabolism, 2004, 48; 404-408

### DISCLOSURE OF THE INVENTION

The object of the present invention is to provide improving agents of side-effects of chemotherapy agents.

The further object of the present invention is to provide pharmaceutical compositions which comprise chemotherapy agents and said compositions wherein the side-effects of the chemotherapy agents are improved.

The inventors focused on erythroid progenitor cells which decrease due to the side-effect of chemotherapy agents, and searched and examined a drug by which the above problems can be solved. As a result, they found that arginine which is an amino acid has the inhibiting action of the decrease in CFU-E of erythroid progenitor cells caused by chemotherapy agents. The present invention has been completed based on this finding.

Examples of the chemotherapy agents used in clinical practice are antimetabolites, e.g. cytosine arabinoside; alkylating agents, e.g. cyclophosphamide; antibiotics of anticancer drugs, e.g. daunorubicin hydrochloride; microtubular agonists, e.g. paclitaxel; irinotecan hydrochloride; cisplatin; and etoposide. For example, the chemotherapy agent VP-16 (etoposide) described in Examples of the present invention is an anticancer drug which has an inhibiting action of topoisomerase II and is applied to lung small cell cancer, malignant lymphoma, uterine cervix cancer, or the like. However, myelosuppression is known as serious side-effects, such as the decrease of leukocytes, the decrease of platelets, and anemia. Therefore, it is required that the observation be carefully made such as frequent blood tests and that suitable treatments be conducted such as the decrease of the administered quantity, suspension of drugs and cessation thereof in case of having abnormalities.

According to the investigation in Japan, because of the use of VP-16, 30.9% of 1648 cases have anemia (the decrease of erythrocytes and hemoglobin) by being administered for 5 consecutive days; and the decrease of hemoglobin is seen in 54.7% of 86 cases by being administered for 21 consecutive days.

Though VP-16 was selected and used as a typical example of chemotherapy agents in Examples of the present invention, the present invention provides improving agents of anemia expressing when using not only VP-16 but also other chemotherapy agents of which cell toxicities against bone-marrow cells are concerned.

More specifically, the in vitro colony assay method wherein a methylcellulose semisolid medium is used is generally used as the method for determining CFU-E. It is the most suitable experimental system for finding the action of improving the decrease of CFU-E caused by chemotherapy agents. The inventors used this method, reacted VP-16 to the isolated mouse bone-marrow cells to conduct myelosuppression, and determined the number of CFU-E colonies when adding 600 μM of arginine. As a result, they found that the number of CFU-E increased by the administration of arginine. In the oral administration to rats, the single administration of 1.2 g/kg of arginine makes the blood concentration thereof reach around 600 μM. Therefore, the increase of CFU-E dependent on the administered dose is expected in the oral administration. In human beings, too, it is expected that the number of CFU-E increases depending on the blood concentration of arginine by the oral administration.

From the above, the inventors found that arginine has the improving action of the side-effects to myelopoiesis caused by the administration of chemotherapy agents, namely, arginine becomes an improving agent of anemia which is excellent in safety and can be orally administered. The present invention has been completed based on this finding.

In addition, arginine improves the CFU-E inhibition of VP-16 even though the sufficient amount of EPO exists. Thus, it is clarified that each EPO and arginine has a different site of action and both have the synergetic effect. Namely, the effects of arginine of the present invention can be used in combination with erythropoietin, erythropoietin mimic peptides, erythropoietin production inducers, or the like.

Now, the present invention provides the followings:

(1) An improving agent of side-effects of chemotherapy agents which comprises arginine as an active ingredient.
(2) The improving agent according to (1), wherein the side-effect of chemotherapy agents is anemia.
(3) The improving agent according to (1) for oral administration.
(4) The improving agent according to (2) for enteral administration.
(5) The improving agent according to any one of (1) to (4), which further combines erythropoietin.
(6) The improving agent according to any one of (1) to (4), which further combines an erythropoietin mimic peptide(s).
(7) The improving agent according to any one of (1) to (4), which further combines an erythropoietin production inducer(s).
(8) The improving agent according to any one of (5) to (7), which is a combination drug.
(9) The improving agent according to (8), which is a kit composed of an agent(s) which comprises arginine and an agent(s) which comprises erythropoietin.
(10) The improving agent according to (8), which is a kit composed of an agent(s) comprising arginine and an agent(s) comprising an erythropoietin mimic peptide(s).
(11) The improving agent according to (8), which is a kit composed of an agent(s) comprising arginine and an agent(s) comprising an erythropoietin production inducer(s).
(12) A pharmaceutical composition which comprises arginine and a chemotherapy agent(s).
(13) The pharmaceutical composition according to (12), wherein each of arginine and the chemotherapy agent is formulated into a separate sterile preparation that can be administered by intravenous drip infusion.
(14) The pharmaceutical composition according to (13), which further comprises erythropoietin, an erythropoietin mimic peptide(s) or an erythropoietin production inducer(s) in the sterile preparation comprising arginine that can be administered by intravenous drip infusion.

The present invention provides improving agents of side-effects of chemotherapy agents which have high safety and versatility, and can be orally administered.

The improving agents of side-effects of chemotherapy agents of the present invention are expected to improve the deterioration of QOL (quality of life) of the patients, mental and physical loss, effects of cancer therapy or the like caused by anemia. In addition to it, it is expected to be able to surely improve anemia if it can be orally administered, because the oral administration does not accompany pain unlike subcutaneous or intravenous injection, and such drug can be easily taken. Further, since the improving agents of side-effects of chemotherapy agents of the present invention comprise an amino acid which exists in the body, there is no expression of the side-effects when taking it. Thus, it is expected to be able to treat anemia without concerning about deterioration of renal function.

### BRIEF DESCRIPTION OF THE DRAWINGS

FIG. 1 shows that, in the existence of the EPO concentration, 0.3 U/mL or more of EPO is the maximal action amount.

FIG. 2 shows the CFU-E inhibiting action of VP-16. More specifically, it shows each CFU-E inhibiting action of 1 nM, 10 nM and 100 nM (final concentration) of VP-16 under the existence of 1 U/mL of EPO, which is the maximal action concentration.

FIG. 3 shows the protecting action of arginine against the CFU-E inhibiting action of VP-16. More specifically, it shows the CFU-E increasing action of arginine under the existences of 1 U/mL of EPO which is the maximal action concentration and 10 nM of VP-16 which has 40% CFU-E inhibiting action. When regarding the number of CFU-E in the control case as 100%, the relative value thereto is shown.

FIG. 4 shows the CFU-E inhibiting action of irinotecan hydrochloride. More specifically, it shows each CFU-E inhibiting action of 1 μM and 10 μM (final concentration) of irinotecan hydrochloride under the existence of 1 U/mL of EPO, which is the maximal action concentration.

FIG. 5 shows the protecting action of arginine against the CFU-E inhibiting action of irinotecan hydrochloride. More specifically, it shows the CFU-E increasing action of arginine under the existences of 1 U/mL of EPO which is the maximal action concentration and 3 μM of irinotecan hydrochloride which has 20% CFU-E inhibiting action. When regarding the number of CFU-E in the control case as 100%, the relative value thereto is shown.

FIG. 6 shows that, in the existence of the EMP1 concentration, 10 μM or more of EMP1 is the maximal action amount.

FIG. 7 shows the protecting action of arginine against the CFU-E inhibiting action of VP-16 under the existence of EMP1. More specifically, it shows the CFU-E increasing action of arginine under the existences of 30 μM of EMP1 which is the maximal action concentration and 10 nM of VP-16. When regarding the number of CFU-E in the control case as 100%, the relative value thereto is shown.

### BEST MODE FOR CARRYING OUT THE INVENTION

The improving agents of side-effects of chemotherapy agents of the present invention comprise arginine as the active ingredient. Arginine preferably includes arginine and physiologically acceptable salts thereof. As for the isomers of the active ingredient, either L-form, D-form or DL-form can be used, and L-form is preferable from the viewpoint that it naturally exists.

The improving agents of side-effects of chemotherapy agents of the present invention can be formulated into various publicly known or possibly developed pharmaceutical preparations, e.g. the administered forms such as oral administration, intraperitoneal administration, transdermal administration, subcutaneous administration, intravenous administration, and inhalation administration. Though arginine can be singularly used in the improving agents of side-effects of chemotherapy agents of the present invention, they can be formulated into various forms as pharmaceutical preparations which comprise arginine as the active ingredient by applying to the publicly known or possibly developed methods, if necessary.

The administration method of the improving agents of side-effects of chemotherapy agents of the present invention is not particularly limited, and the oral administration is preferable. In such a case, the administered dose differs depending on the patient's age, the hemoglobin concentration which becomes the anemia index of the patient, or the like. In case of a normal adult, the dose is about 0.1 to 12 g per a day and preferably about 0.5 to 6 g. It is particularly preferable to use 0.1 to 100 g of arginine per 0.1 g of a chemotherapy agent.

The improving agents of side-effects of chemotherapy agents of the present invention can be used as the active ingredient of pharmaceutical products that are used for treating or preventing the side-effects of chemotherapy agents that induce the decrease of erythroid hematopoiesis; or as the constituent of foods or medical foods.

In the improving agents of side-effects of chemotherapy agents of the present invention, an erythropoietin-like substance(s) can be used in combination with arginine. At that time, it is preferable that 80 to 75,000 IU of an erythropoietin-like substance(s) is used per 1 g of arginine. An erythropoietin-like substance is the substance which exercises or induces the erythropoietin-like actions in vivo when administering/taking such substance in vivo.

For example, erythropoietin-like substances include erythropoietin, erythropoietin mimic peptides, erythropoietin production promoters or the like, but they are not particularly limited to them.

Erythropoietin may be a natural type, genetical recombinant type, or modified type. For example, in the case of the genetical recombinant type, it may be epoetin alfa (genetical recombination) or epoetin beta (genetical recombination), which are glycoproteins (molecular weight: about 30,000) consisting of 165 amino acid residues (C₈₀₉H₁₃₀₁ N₂₂₉O₂₄₀S₅; molecular weight: 18,235.96) produced in Chinese hamster ovary cells by the expression of human erythropoietin cDNA derived from mRNA of human hepatic cells.

An erythropoietin mimic peptide is a peptide which bonds to an EPO receptor and activates it, or works as an EPO agonist. For example, as disclosed in WO96/40749, it includes peptides of 10 to 40 amino acid residues in length, which comprise a sequence of amino acids X3X4GPX6TWX7X8, wherein each amino acid is indicated as the standard one letter abbreviation: X3 is C; X4 is R, H, L or W; X5 is M, F or I; X6 is independently selected from any one of the 20 genetically coded L-amino acids; X7 is D, E, I, L or V; and X8 is C.

More specifically, an EPO mimic peptide includes peptides which comprise the peptides that meet the following requirements as the component(s).

1. A peptide of 10 to 40 amino acid residues in length, which bonds to an EPO receptor and comprises a sequence of amino acids X3X4GPX6TWX7X8 wherein each amino acid is indicated as the standard one letter abbreviation: X6 is independently selected from any one of the 20 genetically coded L-amino acids; X3 is C; X4 is R, H, L or W; X5 is M, F or I; X7 is D, E, I, L or V; and X8 is C.
2. The peptide according to 1, which comprises a sequence of amino acids YX2X3X4X5GPX6TWX7X8 wherein each amino acid is indicated as the standard one letter abbreviation: each X2 and X6 is independently selected from any one of the 20 genetically coded L-amino acids; X3 is C; X4 is R, H, L or W; X5 is M, F or I; X7 is D, E, I, L or V; and X8 is C.
3. The peptide according to 2, which comprises a sequence of amino acids X1YX2X3X4X5GPX6TWX7X8X9X10X11 wherein each amino acid is indicated as the standard one letter abbreviation: each X1, X2, X6, X9, X10 and X11 is independently selected from any one of the 20 genetically coded L-amino acids; X3 is C; X4 is R, H, L or W; X5 is M, F or I; X7 is D, E, I, L or V; and X8 is C.
4. The peptide according to 3, wherein X4 is R or H; X5 is F or M; X6 is I, L, T, M, E or V; X7 is D or V; X9 is G, K, L, Q, R, S or T; and X10 is A, G, P, R or Y.
5. The peptide according to 4, wherein X1 is D, E, L, N, S, T or V; X2 is A, H, K, L, M, S or T; X4 is R or H; X9 is K, R, S or T; and X10 is P.
6. The peptide according to 1, which is selected from the group consisting of the following:

GGLYLCRFGPVTWDCGYKGG;
GGTYSCHFGPLTWVCKPQGG;
GGTYSCHFGPLTWVCKPQ;
GGDYHCRMGPLTWVCKPLGG;
VGNYMCHFGPITWVCRPGGG;
GGNYMCHFGPITWVCRPGGG;
GGVYACRMGPITWVCSPLGG;
VGNYMAHMGPITWVCRPGG;
GGPHHVYACRMGPLTWIC;
GGTYSCHFGPLTWVCKPQ;
GGLYACHMGPMTWVCQPLRG;
TIAQYICYMGPETWECRPSPKA;
YSCHFGPLTWVCK;
TCHFGPLTWVC;
Ac-GGTYSCHFGPLTWVCKPQGG;
GGCRIGPITWVCGG;
LGRKYSCHFGPLTWVCQPAKKD;
GGTASCHFGPLTWVCKPQGG;
GGNYYCRFGPITFECHPTGG;
GGEYLCRMGPMTWVCTPVGG;
GGLYTCRMGPITWVCLPAGG;
GGTTSCHFGPLTWVCKPQGG;
GGTFSCHFGPLTWVCKPQGG;
GGTYSCHFGALTWVCKPQGG;
GGTYSCHFGPLAWVCKPQGG;
GGTYSCHFAPLTWVCKPQGG;
GGTYSCHFGPATWVCKPQGG;
GGTYSCHFGPLTAVCKPQGG;
GGTYSCHFGPLTFVCKPQGG;
TYSCHFGPLTWVCKPQ;
YSCHFGPLTWVCKP;
SCHFGPLTWVCK;
GGTYSCFGPLTWVCKPQGG;
TYSCHFGPLTWVCKPQGG;
YSCHFGPLTWVC;
GGTYSCHFGPLTFVCKPQGG;
and
HFGPLTWV.

7. The peptide according to 1, which is selected from the group consisting of the following:

GGLYLCRFGPVTWDCGYKGG;
GGTYSCHFGPLTWVCKPQGG;
GGDYHCRMGPLTWVCKPLGG;
VGNYMCHFGPITWVCRPGGG;
GGVYACRMGPITWVCSPLGG;
VGNYMAHMGPITWVCRPGG;
GGPHHVYACRMGPLTWIC;
GGTYSCHFGPLTWVCKPQ;
GGLYACHMGPMTWVCQPLRG;
TIAQYICYMGPETWECRPSPKA;
YSCHFGPLTWVCK;
YCHFGPLTWVC;
and
HFGPLTWV.

8. The peptide according to 1, of which the sequence of amino acids is cyclized.
9. The peptide according to 8, which is selected from the group consisting of the following:

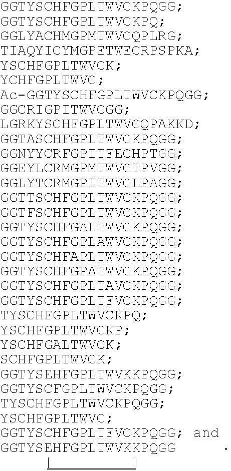

10. The peptide according to 1, of which the sequence of amino acids is dimerized.
11. The peptide according to 10, which comprises the following sequence of amino acids:

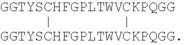

In addition to them, an EPO mimic peptide also includes HEMATIDE™ developed by Affymax, Inc.

In the improving agents of side-effects of chemotherapy agents of the present invention, an EPO production promoter(s) can be used in combination with arginine. At that time, it is preferable that 0.01 to 100 mg of an EPO production promoter(s) is used per 1 g of arginine. An EPO production promoter is a drug which induces the production of biologically-inherent EPO by administering the drug in vivo. For example, it includes a HIF (Hypoxia-Inducible Factor) stabilizer and it may have any structure provided that it has the HIF stabilizing action. More specifically, the example thereof is FG2216 (YM311 by a Japanese name) developed by FibroGen, Inc. Further, it may be an EPO production promoter which has the action mechanism other than that of an inhibitor of hypoxia-inducible factor proline hydroxylase.

An agent which decreases the administered dose of an erythropoietin-like substance is an agent which can decrease the required administered dose of an erythropoietin-like substance.

As the method of applying the active ingredient of the present invention to pharmaceutical products, oral or parenteral administration can be applied. When administering it, the active ingredient can be mixed with a solid or liquid pharmaceutical nontoxic carrier(s) which is suitable for its administration method such as oral administration and injection, and it can be administered in the form of the common pharmaceutical preparation. Examples of such preparations include solid preparations such as tablets, granules, dispersants and capsules; liquid preparations such as solutions, suspension agents and emulsions; and freeze-dry preparations. These preparations can be prepared by the pharmaceutically common method. Examples of the pharmaceutical nontoxic carriers include glucose, lactose, sucrose, starch, mannitol, dextrin, fatty acid glyceride, polyethyleneglycol, hydroxyethyl starch, ethyleneglycol, polyoxyethylene sorbitan fatty acid ester, amino acid, gelatin, albumin, water and normal saline solution. Further, it is possible to add a common additive(s) such as a stabilizer, moisturizer, emulsifying agent, binder and isotonic agent, if necessary.

Arginine is, for example, marketed as soft drinks wherein 1 g of arginine is combined in 100 mL of liquid. As pharmaceutical products, injectable solutions containing 10 W/V % of L-arginine hydrochloride (pH 5.0 to 6.0, osmotic ratio: about 3) are used as agents for urea cycle disorders and stressing agents of pituitary function test (trade name: Argi-U injection by Ajinomoto Co., Inc.; trade name: arginine injection “Ajinomoto” by Ajinomoto Co., Inc.). “Argi-U” injection is an infusion preparation which contains 20.0 g of L-arginine hydrochloride in one plastic infusion bag (200 mL). The infusion bag is put in an oxygen poorly-permeating bag with a deoxidant and sealed to be provided to medical institutions.

As granules containing arginine, an agent for urea cycle disorders is on the market, which combines 605 mg of L-arginine hydrochloride and 500 mg of L-arginine in 1.3 g of the agent. The dosage forms which can be applied to the improving agents of side-effects of chemotherapy agents of the present invention are, for example, the preparations mentioned above.

Meanwhile, in the cancer treatment with chemotherapy agents, the active ingredient may be blended into an infusion such as a normal saline solution and continuously administered by intravenous drip infusion more than 30 minutes. At that time, the effective dose of arginine is taken from an infusion containing 5 to 15% of arginine and added to the blended solution of a chemotherapy agent(s) so that both the chemotherapy agent(s) and arginine can be administered by intravenous drip infusion. Such administration in the form of an infusion is a preferable example of use of the present invention. In addition to it, prefilled syringes wherein a specific amount of an arginine solution is filled and sealed are the more preferable administered form when using arginine in combination with an intravenous drip infusion of a chemotherapy agent(s).

Thus, since the usefulness of the improving agents of side-effects of chemotherapy agents of the present invention is further enhanced by preparing and using them as a sterile preparation that can be administered by intravenous drip infusion, the present invention can provide the anticancer method which comprises the step of administering a chemotherapy agent(s) in combination with arginine. More specifically, the present invention can provide the method of preventing the side-effects of chemotherapy agents which comprises the step of administering both components in the form whereby they can be administered by intravenous drip infusion.

Next, Examples will further illustrate the present invention. They only explain the present invention and do not particularly limit the invention.

### EXAMPLES

### Example 1

### (1) CFU-E Colony Assay

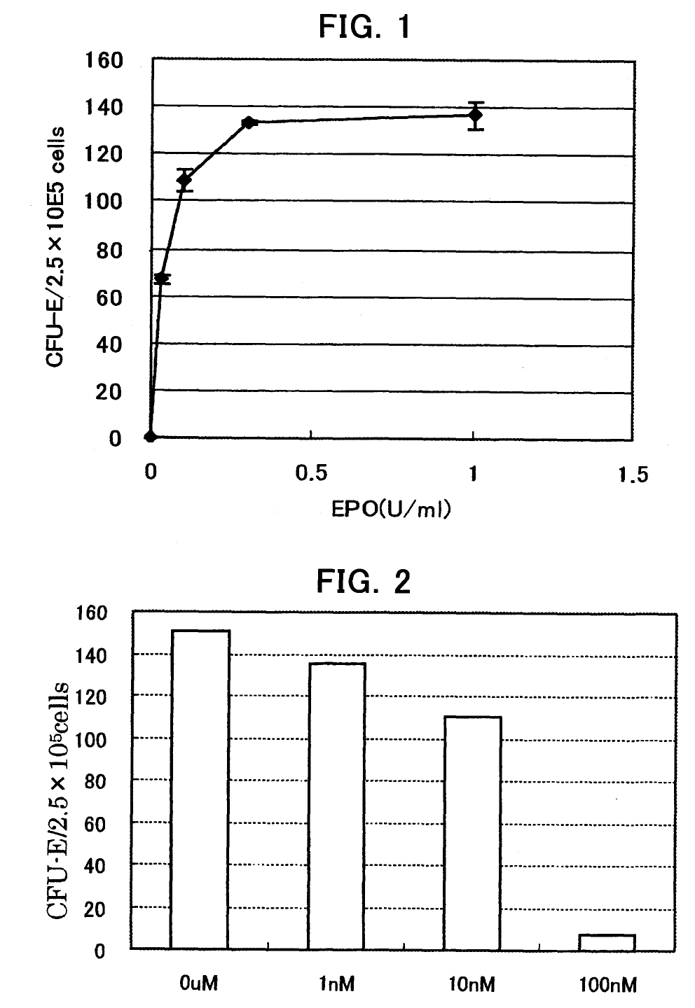

The in vitro CFU-E colony assay was conducted as follows. After making a male BDF-1 mouse of 11 weeks old (by Charles River Laboratories Japan, Inc.) die by the cervical dislocation method, the bone-marrow cells were isolated from the thighbone and suspended in the IMDM medium (by Invitrogen Corporation) which contained 10% FCS (by JRH Biosciences). The bone-marrow cells were centrifuged at 1500 rpm for 10 minutes at 4° C. Then, the precipitated bone-marrow cells were substituted on the IMDM medium without an amino acid, and the number of the cells was determined. 1 mL of a methylcellulose semisolid medium wherein the bone-marrow cells were suspended in the IMDM medium of ⅓ concentration (0.03, 0.1, 0.3 and 1.0 U/mL of rHuEPO by Chugai Pharmaceutical Co., Ltd.; 100 μM of 2-mercaptoethanol by Wako Pure Chemical Industries, Ltd.; 15% FCS by JRH Biosciences; 0.8% methylcellulose, IMDM solution M3134 containing methylcellulose by StemCell Technologies Inc.; 1% BSA by Sigma-Aldrich Japan K.K.; and bone-marrow cells 2.5×10⁵/mL) was added to a dish of 3.5 cm in diameter (Nalge Nunc International K.K.). After the medium was cultured at 37° C. under 5% CO₂ for 48 hours, the number of CFU-E colonies was determined by using an inverted microscope. FIG. 1 shows the result of the CFU-E colony assay. Data is indicated as the average value.

### (2) CFU-E Inhibiting Action of the Chemotherapy Agent Etoposide (VP-16)

The in vitro CFU-E colony assay was conducted in the EPO concentration of the maximal action amount thereof. In addition to the composition of the methylcellulose semisolid medium wherein the bone-marrow cells were suspended in the IMDM medium of ⅓ concentration prepared by the same method as that of Example 1, the mediums to which the chemotherapy agent etoposide (VP-16) was further added so that the final concentration became 1 nM, 10 nM and 100 nM were prepared and cultured at 37° C. under 5% CO₂ for 48 hours. Then, the number of CFU-E colonies was determined by using an inverted microscope. FIG. 2 shows the result of the CFU-E colony assay. Data is indicated as the average value.

As clarified in FIG. 2, 100 nM of VP-16 nearly completely inhibited the expression of CFU-E colonies, 10 nM thereof inhibited the expression by 30% and 1 nM thereof inhibited the expression by 10%. Thus, the number of CFU-E colonies decreased depending on the concentration of VP-16. Accordingly, it was confirmed that VP-16 directly acts on the bone-marrow cells and decreases the number of CFU-E, that is, it has the inhibiting action against erythroid hematopoiesis.

### (3) The Improving Effect of Arginine on the CFU-E Inhibiting Action of VP-16

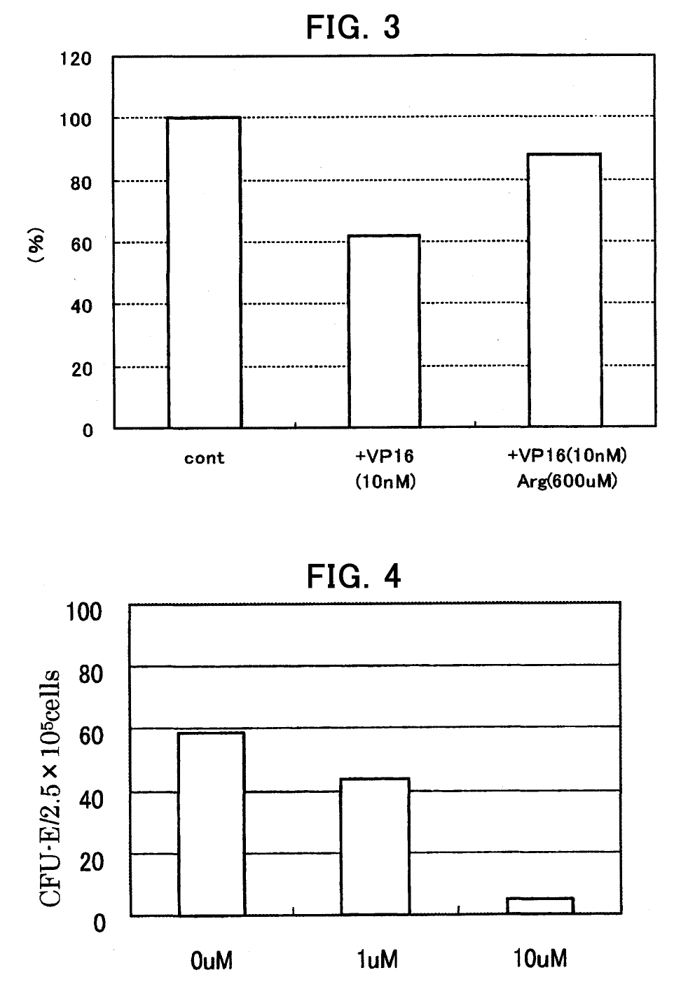

The CFU-E colony assay was conducted by the same method as that of Example 1. The medium to which only 10 nM of VP-16 in its final concentration was added and the medium to which VP-16 in the same concentration as above and 600 uM of arginine were simultaneously further added were prepared and the number of CFU-E colonies was determined. FIG. 3 shows the result of the CFU-E colony assay. Data is indicated as the average value. The vertical axis is the relative value (%) when regarding the number of CFU-E colonies in the control case as 100%.

As clarified in FIG. 3, it was confirmed that the number of CFU-E colonies which decreased by about 40% due to the single addition of VP-16 was improved by almost 90% in case of adding 600 uM of arginine simultaneously. This clarified that arginine has the improving effect against the CFU-E inhibiting action of chemotherapy agents such as VP-16, that is, it improves anemia caused by the side-effects of chemotherapy agents.

As FIG. 1 shows, EPO increases the erythroid stem cell CFU-E depending on its concentration, and it becomes the maximal activity when each reaches the specified concentration. After that, the maximal activity is kept though the concentration increases. The effect of arginine mentioned in the present invention further increases CFU-E when acting it simultaneously even though such sufficient amount of EPO exists. Thus, it is clarified that each EPO and arginine has a different site of action and both have the synergetic effect.

### Example 2

### An Aqueous Injectable Solution Containing L-Arginine Hydrochloride

L-arginine hydrochloride is dissolved in distilled water for injection so that its concentration becomes 10 w/v % and prepared. Then, when filtering and sterilizing it at 105° C. for 25 minutes, pH is 5.8 and osmotic ratio is 2.7.

### Example 3

### (1) CFU-E Inhibiting Action of the Chemotherapy Agent Irinotecan Hydrochloride

The in vitro CFU-E colony assay was conducted in the EPO concentration of the maximal action amount thereof. The bone-marrow cells were prepared by the same method as that of Example 1, using a male BDF-1 mouse of 12 weeks old (by Charles River Laboratories Japan, Inc.). Irinotecan hydrochloride was further added to a methylcellulose semisolid medium wherein the bone-marrow cells were suspended in the IMDM medium of ⅓ concentration (1.0 U/mL of rHuEPO by Chugai Pharmaceutical Co., Ltd.; 100 μM of 2-mercaptoethanol by Wako Pure Chemical Industries, Ltd.; 30% FCS by JRH Biosciences; 0.8% methylcellulose, IMDM solution M3134 containing methylcellulose by StemCell Technologies Inc.; 1% BSA by Sigma-Aldrich Japan K.K.; and bone-marrow cells 2.5×10⁵/mL), so that the final concentration of irinotecan hydrochloride became 1 μM and 10 μM. After the medium was cultured at 37° C. under 5% CO₂ for 48 hours, the number of CFU-E colonies was determined by using an inverted microscope. FIG. 4 shows the result of the CFU-E colony assay. Data is indicated as the average value.

As clarified in FIG. 4, 10 μM of irinotecan hydrochloride nearly completely inhibited the expression of CFU-E colonies, and 1 μM thereof inhibited the expression of CFU-E colonies by 30%. Thus, it was confirmed that irinotecan hydrochloride directly acts on the bone-marrow cells and decreases the number of CFU-E, that is, it has the inhibiting action against erythroid hematopoiesis.

### (2) The Improving Effect of Arginine on the CFU-E Inhibiting Action of Irinotecan Hydrochloride

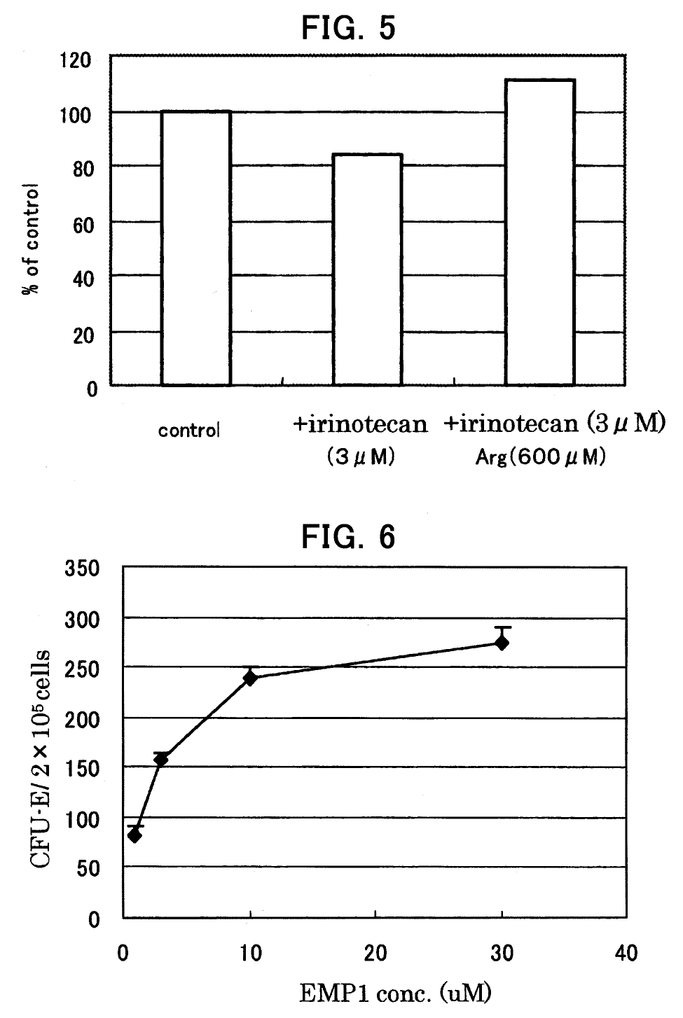

The CFU-E colony assay was conducted by the same method as that of above (1), using a male BDF-1 mouse of 11 weeks old (by Charles River Laboratories Japan, Inc.). The medium to which only 3 μM of irinotecan hydrochloride in its final concentration was added and the medium to which irinotecan hydrochloride in the same concentration as above and 600 uM of arginine were simultaneously further added were prepared and the number of CFU-E colonies was determined. FIG. 5 shows the result of the CFU-E colony assay. Data is indicated as the average value. The vertical axis is the relative value (%) when regarding the number of CFU-E colonies in the control case as 100%.

As clarified in FIG. 5, it was confirmed that the number of CFU-E colonies which decreased by about 20% due to the single addition of irinotecan hydrochloride increased by almost 30% in case of adding 600 uM of arginine simultaneously, as compared with the value of the single addition of irinotecan hydrochloride. This clarified that arginine also has the improving effect against the CFU-E inhibiting action of irinotecan hydrochloride as well as VP-16, that is, it improves anemia caused by the side-effects of multiple chemotherapy agents.

### Example 4

### (1) CFU-E Colony Assay Under the Existence of an EPO Mimic Peptide (EMP1)

As a typical example of an EPO mimic peptide, EMP1 (GGTYSCHFGPLTWVCKPQGG-NH₂, C₆-C₁₅ disulfide bonding) described in the literature (Table 1 of Science Vol. 273, 458-463, 1996, etc) was prepared by using the method described in the literature (3700-3701 pages of Biochemistry Vol. 37(11), 3699-3710).

The in vitro CFU-E colony assay was conducted as follows.

The bone-marrow cells were prepared by the same method as that of Example 1, using a male BDF-1 mouse of 10 weeks old (by Charles River Laboratories Japan, Inc.). 1 mL of a methylcellulose semisolid medium wherein the bone-marrow cells were suspended in the IMDM medium of ⅓ concentration (1, 3, 10 and 30 μM of EMP1; 100 μM of 2-mercaptoethanol by Wako Pure Chemical Industries, Ltd.; 24% FCS by JRH Biosciences; 0.8% methylcellulose, IMDM solution M3134 containing methylcellulose by StemCell Technologies Inc.; 2% BSA by Sigma-Aldrich Japan K.K.; and bone-marrow cells 2×10⁵/mL) was added to a dish of 3.5 cm in diameter (Nalge Nunc International K.K.). After the medium was cultured at 37° C. under 5% CO₂ for 48 hours, the number of CFU-E colonies was determined by using an inverted microscope. FIG. 6 shows the result of the CFU-E colony assay. Data is indicated as the average value.

### (2) The Improving Effect of Arginine on the CFU-E Inhibiting Action of VP-16 Under the Existence of EMP1

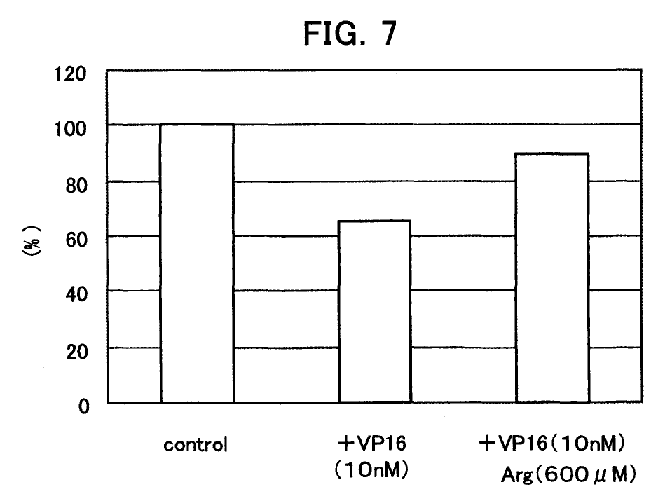

The in vitro CFU-E colony assay was conducted in the EMP1 concentration of the maximal action amount thereof. The bone-marrow cells were prepared by the same method as that of Example 1, using a male BDF-1 mouse of 10 weeks old (by Charles River Laboratories Japan, Inc.). Then, prepared were the medium wherein only 10 nM of VP-16 in its final concentration was further added to the methylcellulose semisolid medium wherein the bone-marrow cells were suspended in the IMDM medium of ⅓ concentration (30 μM of EMP1; 100 μM of 2-mercaptoethanol by Wako Pure Chemical Industries, Ltd.; 30% FCS by JRH Biosciences; 0.8% methylcellulose, IMDM solution M3134 containing methylcellulose by StemCell Technologies Inc.; 1% BSA by Sigma-Aldrich Japan K.K.; and bone-marrow cells 2.5×10⁵/mL); and the medium wherein VP-16 in the same concentration as above and 600 μM of arginine were simultaneously further added to said methylcellulose semisolid medium. After each medium was cultured at 37° C. under 5% CO₂ for 48 hours, the number of CFU-E colonies was determined by using an inverted microscope. FIG. 7 shows the result of the CFU-E colony assay. Data is indicated as the average value. The vertical axis is the relative value (%) when regarding the number of CFU-E colonies in the control case as 100%.

As clarified in FIG. 7, it was confirmed that the number of CFU-E colonies which decreased by about 30% due to the single addition of VP-16 was improved by almost 90% in case of adding 600 μM of arginine simultaneously. This clarified that arginine also has the improving effect against the CFU-E inhibiting action of chemotherapy agents such as VP-16 under the existence of EMP1, that is, it improves anemia caused by the side-effects of chemotherapy agents.

As FIG. 6 shows, EMP1 increases the erythroid stem cell CFU-E depending on its concentration, and it becomes the maximal activity when each reaches the specified concentration. After that, the maximal activity is kept though the concentration increases. The effect of arginine mentioned in the present invention further increases CFU-E when acting it simultaneously even though such sufficient amount of EMP1 exists. Thus, it is clarified that each arginine and EMP1, which is an EPO mimic peptide, has a different site of action and both have the synergetic effect.

What is claimed is:

1. An improving agent of side-effects of chemotherapy agents which comprises arginine as an active ingredient.

2. The improving agent according to claim 1, wherein the side-effect of chemotherapy agents is anemia.

3. The improving agent according to claim 1 for oral administration.

4. The improving agent according to claim 2 for enteral administration.

5. The improving agent according to claim 1, which further combines erythropoietin.

6. The improving agent according to claim 1, which further combines an erythropoietin mimic peptide(s).

7. The improving agent according to claim 1, which further combines an erythropoietin production inducer(s).

8. The improving agent according to claim 5, which is a combination drug.

9. The improving agent according to claim 8, which is a kit composed of an agent(s) which comprises arginine and an agent(s) which comprises erythropoietin.

10. The improving agent according to claim 8, which is a kit composed of an agent(s) which comprises arginine and an agent(s) which comprises an erythropoietin mimic peptide(s).

11. The improving agent according to claim 8, which is a kit composed of an agent(s) which comprises arginine and an agent(s) which comprises an erythropoietin production inducer(s).

12. A pharmaceutical composition which comprises arginine and a chemotherapy agent(s).

13. The pharmaceutical composition according to claim 12, wherein each of arginine and the chemotherapy agent is formulated into a separate sterile preparation that can be administered by intravenous drip infusion.

14. The pharmaceutical composition according to claim 13, which further comprises erythropoietin, an erythropoietin mimic peptide(s) or an erythropoietin production inducer(s) in the sterile preparation comprising arginine that can be administered by intravenous drip infusion.
